(19) United States
(12) Patent Application Publication
(10) Pub. No.: US 2012/0321658 A1
(43) Pub. Date: Dec. 20, 2012

(54) IMMUNOGENIC COMPOSITION COMPRISING S. PNEUMONIAE POLYSACCHARIDES CONJUGATED TO CARRIER PROTEINS

(76) Inventors: Ralph Leon Biemans, Rixensart (BE); Pierre Duvivier, Rixensart (BE); Ollivier Francis Nicolas Gavard, Rixensart (BE); Jan Poolman, Rixensart (BE)

(21) Appl. No.: 13/581,686

(22) PCT Filed: Aug. 17, 2010
(86) PCT No.: PCT/EP2010/061963

§ 371 Date: Aug. 29, 2012

(30) Foreign Application Priority Data
Mar. 9, 2010 (GB) 10039247.6

(51) Int. Cl. C07K 17/10; A61P 37/04; A61K 39/385

(52) U.S. Cl. 424/194.1; 530/395

(57) ABSTRACT

The present invention relates to an immunogenic composition comprising at least 2 different S. pneumoniae capsular saccharides, wherein one or more is/are selected from a first group consisting of serotypes 1, 3, 19A and 19F which is/are linked to a protein carrier(s) either directly or indirectly through a chemistry other than reductive amination, and one or more different saccharides is/are selected from a second group consisting of serotypes 4, 5, 6A, 6B, 7F, 9V, 14, 18C and 23F which is/are linked to a protein carrier(s) by reductive amination. Uses of such compositions are also disclosed.

The present invention relates to the field of pneumococcal conjugate immunogenic compositions or vaccines wherein different conjugation chemistries are used for different components of the immunogenic composition or vaccine. Reductive amination is used for the conjugation of at least one serotype and a conjugation other than reductive amination is used for the conjugation of a different serotype. The present invention also relates to methods of manufacturing such vaccines and their use in therapy.

Children less than 2 years of age do not mount an immune response to most polysaccharide vaccines, so it has been necessary to render the polysaccharides immunogenic by chemical conjugation to a protein carrier. Coupling the polysaccharide, a T-independent antigen, to a protein, a T-dependent antigen, confers upon the polysaccharide the properties of T dependency including isotype switching, affinity maturation, and memory induction.

Streptococcus pneumoniae is a Gram-positive bacterium responsible for considerable morbidity and mortality (particularly in the young and aged), causing invasive diseases such as pneumonia, bacteraemia and meningitis, and diseases associated with colonisation, such as acute Otitis media. The rate of pneumococcal pneumonia in the US for persons over 60 years of age is estimated to be 3 to 8 per 100,000. In 20% of cases this leads to bacteraemia, and other manifestations such as meningitis, with a mortality rate close to 30% even with antibiotic treatment.

Pneumococcus is encapsulated with a chemically linked polysaccharide which confers serotype specificity. There are 90 known serotypes of pneumococci, and the capsule is the principle virulence determinant for pneumococci, as the capsule not only protects the inner surface of the bacteria from complement, but is itself poorly immunogenic. Polysaccharides are T-independent antigens, and can not be processed or presented on MHC molecules to interact with T-cells. They can however, stimulate the immune system through an alternate mechanism which involves cross-linking of surface receptors on B cells.

It was shown in several experiments that protection against invasive pneumococci disease is correlated most strongly with antibody specific for the capsule, and the protection is serotype specific.

Streptococcus pneumoniae is the most common cause of invasive bacterial disease and Otitis media in infants and young children. Likewise, the elderly mount poor responses to pneumococcal vaccines [Roghmann et al., (1987), J. Gerontol. 42:265-270], hence the increased incidence of bacterial pneumonia in this population [Verghese and Berk, (1983) Medicine (Baltimore) 62:271-285].

Multivalent pneumococcal conjugate vaccines have been developed. Synflorix is marketed by Glaxosmithkline Biological s.a. and contains pneumococcal serotypes 1, 4, 5, 6B, 7F, 9V, 14, and 23F polysaccharides conjugated to protein D from Haemophilus influenzae, 18C conjugated to tetanus toxoid and 19F conjugated to diphtheria toxoid via cyanylation (CDAP) chemistry. Prevenar is marketed by Pfizer and contains pneumococcal serotypes 4, 6B, 9V, 14, 18C, 19F and 23F all conjugated to the non-toxic diphtheria toxoin CRM197 by reductive amination chemistry (Prymula and Schuerman Expert Rev. vaccines 8; 1479-1500 (2009)).

It is an object of the present invention to develop an improved formulation of a multiple serotype Streptococcus pneumoniae polysaccharide conjugate vaccine. This can be achieved by combining saccharides from different pneumococcal serotypes which have been conjugated using different conjugation methods. In this way, the optimum conjugation method is selected for different serotypes allowing each serotype to be presented using a conjugation method that allows the best presentation of the saccharide epitope. Whereas some pneumococcal saccharides conjugate well using reductive amination, for other pneumococcal saccharides, different conjugation methods allow the ring structure to remain unbroken and can provide better results. The selection of which saccharides perform best using either reductive amination or other conjugation methods allows a more effective immunogenic composition to be developed.

Accordingly there is provided an immunogenic composition comprising at least 2 different S. pneumoniae capsular saccharides, wherein one or more is/are selected from a first group consisting of serotypes 1, 3, 19A and 19F which is/are linked to a protein carrier(s) either directly or indirectly through a chemistry other than reductive amination, and one or more different saccharides is/are selected from a second group consisting of serotypes 4, 5, 6A, 6B, 7F, 9V, 14, 18C and 23F which is/are linked to a protein carrier(s) by reductive amination

### BRIEF DESCRIPTION OF FIGURES

FIG. 1. Preparation of polysaccharide-protein conjugates.

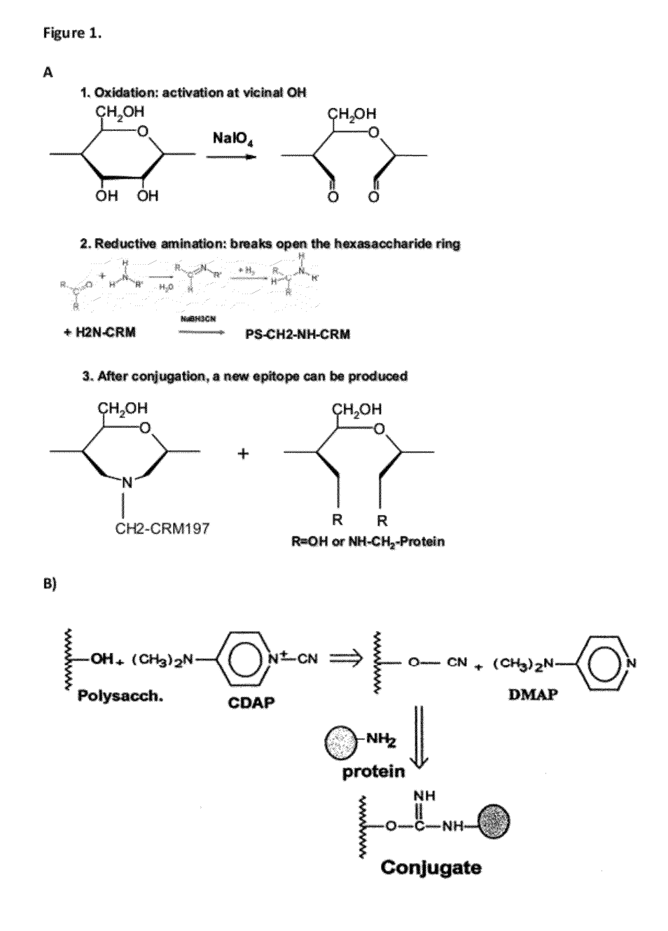

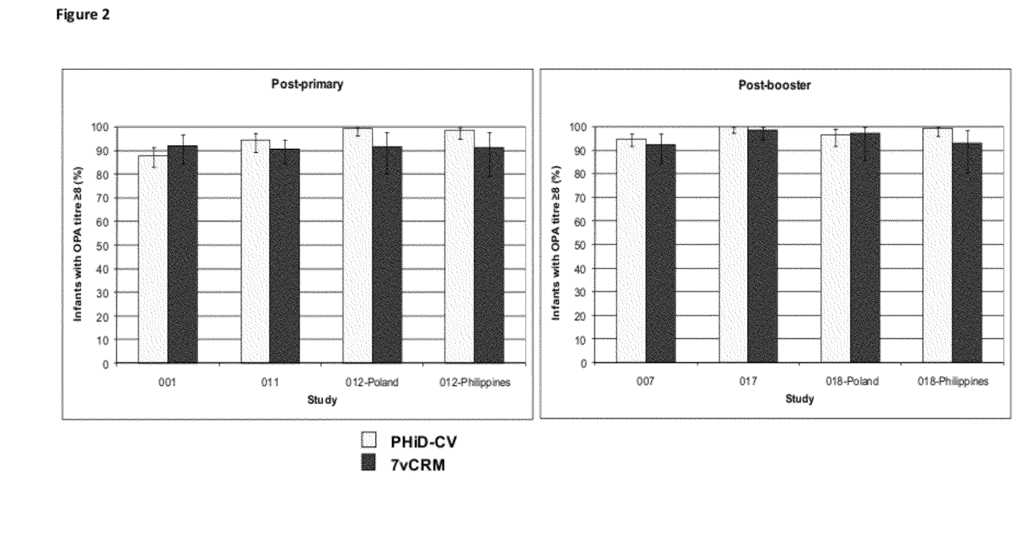

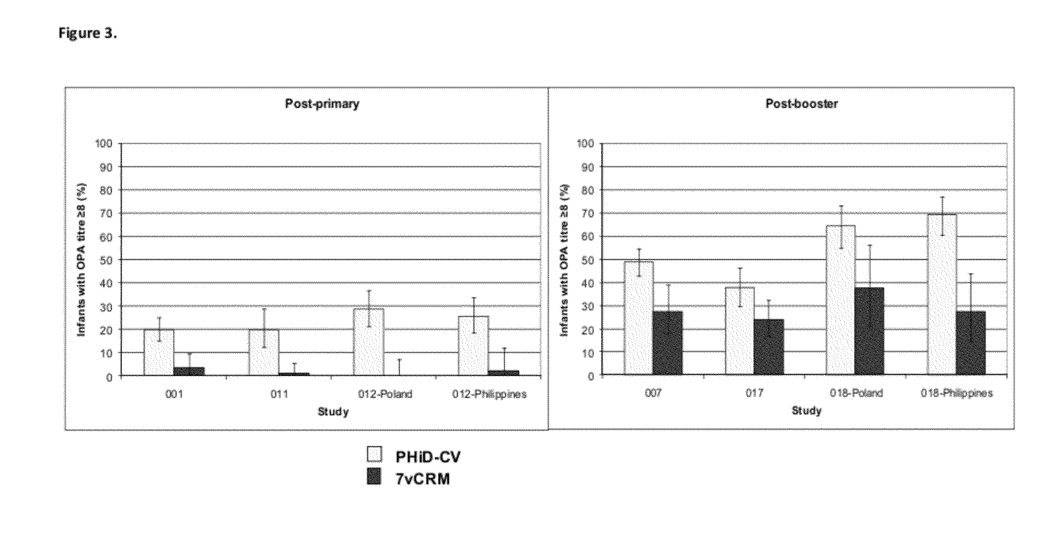

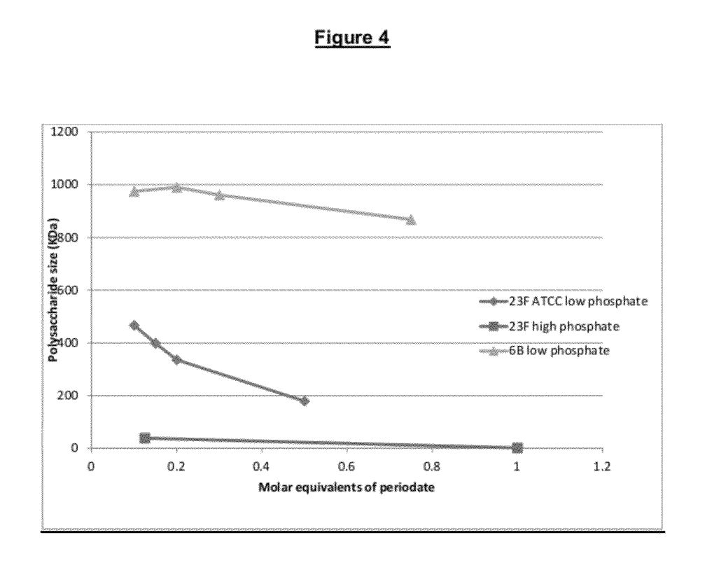

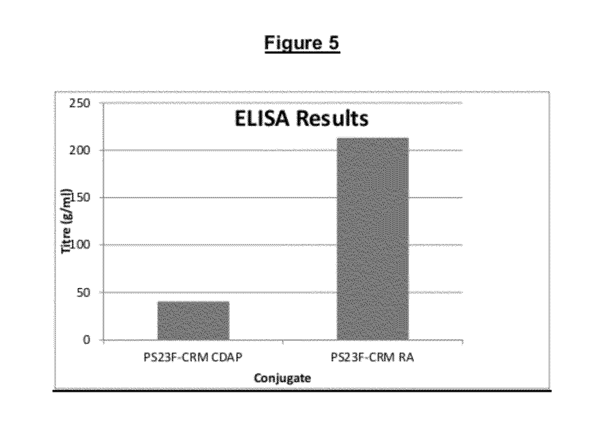

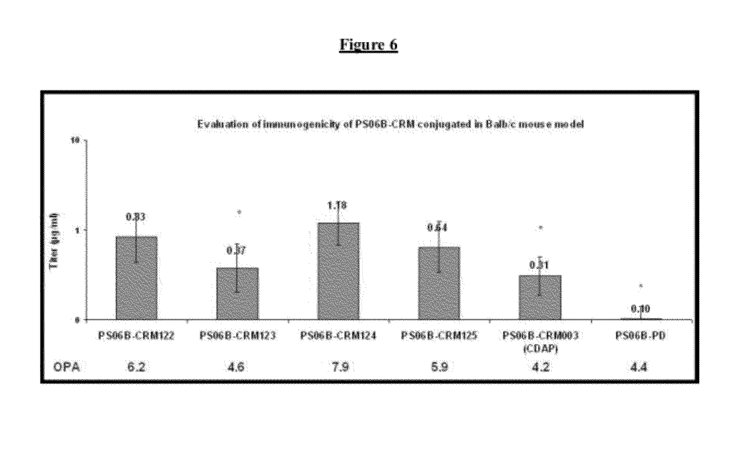

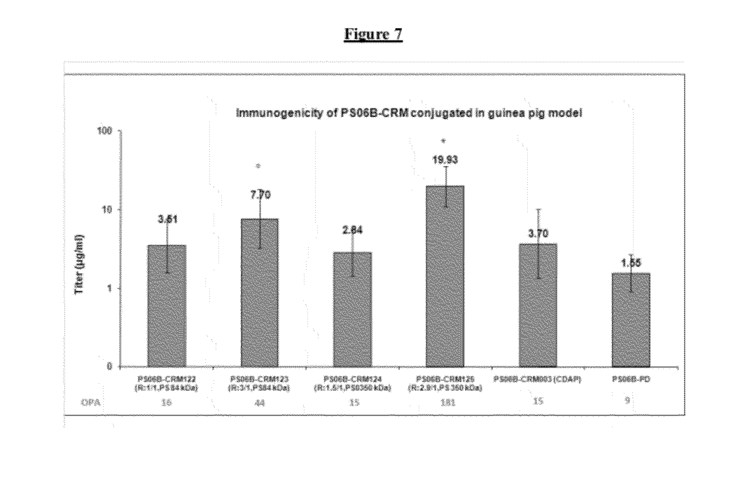

A) In 7vCRM, the 19F polysaccharide is conjugated to the non-toxic diphtheria CRM₁₉₇ protein via reductive amination. (1) oxidation with periodate introduces terminal reactive aldehydes. (2) linkage to the CRM₁₉₇ carrier protein by reductive amination breaks and opens the hexasaccharide ring. (3) After conjugation, a new immunogenic epitope can be produced due to binding of new groups to the hexasaccharide ring.

B) In PHiD-CV, the 19F polysaccharide is conjugated to diphtheria toxoid via cyanylation chemistry. 19F is chemically activated to introduce a cyanate group to the hydroxyl group, forming a covalent bond to the amino or hydrazide group upon addition of the protein component. After cyanylation conjugation the hexasaccharide ring remains intact and other chemical groups are not able to bind.

FIG. 2. Proportion of infants achieving OPA titres≧8 against pneumococcal serotype 19F following PHiD-CV or 7vCRM primary and booster immunisation. Light bars show results for PHiD-CV and dark bars show results for 7vCRM. Error bars represent 95% Confidence limits.

FIG. 3. Proportion of infants achieving OPA titres≧8 against pneumococcal serotype 19A following PHiD-CV or 7vCRM primary and booster immunisation. Light bars show results for PHiD-CV and dark bars show results for 7vCRM. Error bars represent 95% confidence limits.

FIG. 4. Size of 23F and 6B polysaccharides following periodate treatment. The line marked with triangles shows the size of 6B in 10 mM phosphate buffer, the line marker with diamonds shows the size of 23F in 10 mM phosphate buffer and the line marked with squares shows the size of 23F in 100 mM phosphate buffer.

FIG. 5. Comparison of immunogenicity of 23F conjugates using either CDAP or reductive amination conjugation.

FIG. 6. Comparison of immunogenicity in mice of 6B conjugates made by reductive amination or CDAP. The graph shows ELISA titres of four conjugates made by reduction amination (PS06B-CRM122-125) and two made by CDAP (PS06B-CRM003 and PS06B-PD). OPA results are shown below.

FIG. 7. Comparison of immunogenicity in guinea pigs of 6B conjugates made by reductive amination or CDAP. The graph shows ELISA titres of four conjugates made by reduction amination (PS06B-CRM122-125) and two made by CDAP (PS06B-CRM003 and PS06B-PD). OPA results are shown below.

### DESCRIPTION OF THE INVENTION

The present invention provides an immunogenic composition comprising at least 2, 3, 4, 5, 6, 7, 8, 9, 10, 11, 12, 13, 14, 15, 16, 17, 18, 19 or 20 different S. pneumoniae capsular saccharides, wherein one or more is/are selected from a first group consisting of serotypes 1, 3, 19A and 19F which is/are linked to a protein carrier(s) either directly or indirectly through a chemistry other than reductive amination, and one or more different saccharides is/are selected from a second group consisting of serotypes 4, 5, 6A, 6B, 6C, 7F, 9V, 14, 18C and 23F which is/are linked to a protein carrier(s) by reductive amination.

In an embodiment, the immunogenic composition of the invention comprises S. pneumoniae capsular saccharide(s) from serotype 1 or 3 or 19A or 19F; 1 and 3; 1 and 19A; 1 and 19F; 3 and 19A; 3 and 19F; 19A and 19F; 1, 3 and 19A; 1, 3 and 19F, 1, 19A and 19F; 3, 19A and 19F or 1, 3, 19A and 19F conjugated to a protein carrier through a chemistry other than reductive animation. In an embodiment, 19F is conjugated to a carrier protein through a chemistry other than reductive amination.

Optionally capsular saccharide from serotype 1, 3, 19A or 19F are conjugated using reductive amination as long as a further member of this group is conjugated using a method other than reductive amination.

In an embodiment, the immunogenic composition of the invention comprises S. pneumoniae capsular saccharide from serotype 1 or 3 or 19A or 19F; 1 and 3; 1 and 19A; 1 and 19F; 3 and 19A; 3 and 19F; 19A and 19F; 1, 3 and 19A; 1, 3 and 19F, 1, 19A and 19F; 3, 19A and 19F or 1, 3, 19A and 19F conjugated to a protein carrier through cyanylation chemistry such as CDAP chemistry. In an embodiment, 19F is conjugated to a carrier protein by CDAP chemistry.

In an embodiment, the immunogenic composition of the invention comprises S. pneumoniae capsular saccharide from serotype 1 or 3 or 19A or 19F; 1 and 3; 1 and 19A; 1 and 19F; 3 and 19A; 3 and 19F; 19A and 19F; 1, 3 and 19A; 1, 3 and 19F, 1, 19A and 19F; 3, 19A and 19F or 1, 3, 19A and 19F conjugated to a protein carrier through carbodiimide, for example EDAC, chemistry.

In an embodiment of the invention, the following S. pneumoniae capsular saccharide or group thereof is conjugated to a carrier protein by reductive amination; serotype 4, 5, 6A, 6B, 7F, 9V, 14, 18C or 23F, 4 and 5, 4 and 6A, 4 and 6B, 4 and 7F, 4 and 9V, 4 and 14, 4 and 18C, 4 and 23F, 5 and 6A, 5 and 6B, 5 and 7F, 5 and 9V, 5 and 14, 5 and 18C, 5 and 23F, 6A and 6B, 6A and 7F, 6A and 9V, 6A and 14, 6A and 18C, 6A and 23F, 6B and 7F, 6B and 9V, 6B and 14, 6B and 18C, 6B and 23F, 7F and 9V, 7F and 14, 7F and 18C, 7F and 23F, 9V and 14, 9V and 18C, 9V and 23F, 14 and 18C, 14 and 23F or 18C and 23F. In an embodiment, 23F is conjugated to a carrier protein by reductive amination chemistry.

In an embodiment of the invention, the pneumococcal polysaccharide from serotype 19F is conjugated to a carrier protein by cyanylation chemistry for example CDAP chemistry while the pneumococcal polysaccharide from serotype 23 is conjugated to a carrier protein by reductive amination chemistry.

In an embodiment of the invention, the pneumococcal polysaccharide from serotype 19F is conjugated to a carrier protein by cyanylation chemistry for example CDAP chemistry while the pneumococcal polysaccharide from serotype 6B is conjugated to a carrier protein by reductive amination chemistry.

In an embodiment of the invention, the pneumococcal polysaccharide from serotype 19F is conjugated to a carrier protein by cyanylation chemistry for example CDAP chemistry while the pneumococcal polysaccharide from serotype 6A is conjugated to a carrier protein by reductive amination chemistry.

In an embodiment of the invention, the pneumococcal polysaccharide from serotype 19F is conjugated to a carrier protein by cyanylation chemistry for example CDAP chemistry while the pneumococcal polysaccharide from serotype 6C is conjugated to a carrier protein by reductive amination chemistry.

In an embodiment of the invention, 1, 2, 3, 4, 5, 6, 7, 8, 9, 10, 11 or 12 S. pneumoniae capsular sacharides from different serotypes are conjugated to a carrier protein using reductive amination chemistry.

Where reductive amination chemistry is used to conjugate S. pneumoniae capsular saccharides are optionally oxidised using 0.1-1.2, 0.1-0.5, 0.1-0.2, 0.5-0.8, 0.1-0.8, 0.3-1.0 or 0.4-0.9 molar equivalents of periodate to form an activated saccharide. Optionally the periodate treatment step is carried out in a buffer which does not contain an amine group, for example phosphate buffer, borate buffer, acetate buffer, carbonate buffer and citrate buffer. In an embodiment, the buffer is an inorganic buffer. In an embodiment, the buffer is a phosphate buffer, for example a sodium phosphate buffer or a potassium phosphate buffer. The inventors have noted that by controlling the conditions of the oxidation step of the reductive amination process, the resultant conjugates can advantageously retain size and/or immunogenicity of the saccharide.

In an embodiment, the buffer, for example a phosphate buffer, has a concentration between 1-100 mM, 5-80 mM, 1-50 mM, 1-25 mM, 10-40 mM, 1-10 mM, 5-15 mM, 8-12 mM, 10-20 mM, 5-20 mM, 10-50 mM, around 10 mM or around 20 mM. In an embodiment the pH of the buffer is pH 5.0-7.0, pH 5.5-6.5, pH 5.8-6.3, or around pH 6.0.

The term ‘periodate’ includes both periodate and periodic acid. This term also includes both meta periodate (IO₄⁻) and orthoperiodate (IO₆⁵⁻), however in one particular embodiment the periodate used in the method of the invention is metaperiodate. The term ‘periodate’ also includes the various salts of periodate including sodium periodate and potassium periodate. When an antigen reacts with periodate, periodate oxidises vicinal hydroxyl groups to form carbonyl or aldehyde groups and causes cleavage of a C—C bond. For this reason the term ‘reacting an antigen with periodate’ includes oxidation of vicinal hydroxyl groups by periodate, for example the reaction may involve oxidation of cis or trans vicinal diols.

In an embodiment of the invention, 1, 2, 3, 4, 5, 6, 7, 8, 9, 10, 11 or 12 S. pneumoniae capsular saccharides from different serotypes are conjugated to a carrier protein using CDAP chemistry.

In an embodiment of the invention, 1, 2, 3, 4, 5, 6, 7, 8, 9, 10, 11 or 12 S. pneumoniae capsular saccharides from different serotypes are conjugated to a carrier protein using carbodiimide, for example EDAC, chemistry.

In an embodiment, the immunogenic composition of the invention contains a carrier protein selected from the group consisting of tetanus toxoid, diphtheria toxoid, CRM197, Protein D, pneumolysin and PhtD or fragments or fusion proteins thereof.

In an embodiment, the immunogenic composition of the invention contains 2, 3, 4, 5, 6 or 7 different carrier proteins which are separately conjugated to at least or exactly 2, 3, 4, 5, 6, 7, 8, 9, 10, 11, 12, 13, 14, 15 or 16 different S. pneumoniae capsular saccharide serotypes. Optionally these carrier proteins are selected from the group consisting of tetanus toxoid, diphtheria toxoid, CRM197, Protein D, pneumolysin and PhtD or fragments or fusion proteins thereof.

In an embodiment, the immunogenic composition of the invention comprises S. pneumoniae capsular saccharide 1 conjugated to protein D or CRM197.

In an embodiment, the immunogenic composition of the invention comprises S. pneumoniae capsular saccharide 3 conjugated to protein D, CRM197, pneumolysin or PhtD or fragment or fusion protein thereof.

In an embodiment, the immunogenic composition of the invention comprises S. pneumoniae capsular saccharide 4 conjugated to protein D or CRM197.

In an embodiment, the immunogenic composition of the invention comprises S. pneumoniae capsular saccharide 5 conjugated to protein D or CRM197.

In an embodiment, the immunogenic composition of the invention comprises S. pneumoniae capsular saccharide 6B conjugated to protein D or CRM197.

In an embodiment, the immunogenic composition of the invention comprises S. pneumoniae capsular saccharide 7F conjugated to protein D or CRM197.

In an embodiment, the immunogenic composition of the invention comprises S. pneumoniae capsular saccharide 9V conjugated to protein D or CRM197.

In an embodiment, the immunogenic composition of the invention further comprises S. pneumoniae capsular saccharide 14 conjugated to protein D or CRM197.

In an embodiment, the immunogenic composition of the invention comprises S. pneumoniae capsular saccharide 23F conjugated to protein D or CRM197.

In an embodiment, the immunogenic composition of the invention comprises S. pneumoniae capsular saccharide 18C conjugated to tetanus toxoid or CRM197.

In an embodiment, the immunogenic composition of the invention comprises S. pneumoniae capsular saccharide 19A conjugated to pneumolysin or CRM197.

In an embodiment, the immunogenic composition of the invention comprises S. pneumoniae capsular saccharide 22F conjugated to CRM197 or PhtD or fragment of fusion protein thereof.

In an embodiment, the immunogenic composition of the invention comprises S. pneumoniae capsular saccharide 6A conjugated to pneumolysin or a H. influenzae protein, optionally protein D or PhtD or fusion protein thereof or CRM197.

In an embodiment, the immunogenic composition of the invention comprises S. pneumoniae capsular saccharide 6C conjugated to pneumolysin or a H. influenzae protein, optionally protein D or PhtD or fusion protein thereof or CRM197.

The term “saccharide” throughout this specification may indicate polysaccharide or oligosaccharide and includes both. Polysaccharides are isolated from bacteria and may be sized to some degree by known methods (see for example EP497524 and EP497525) and optionally by microfluidisation. Polysaccharides can be sized in order to reduce viscosity in polysaccharide samples and/or to improve filterability for conjugated products. Oligosaccharides have a low number of repeat units (typically 5-30 repeat units) and are typically hydrolysed polysaccharides

Capsular polysaccharides of Streptococcus pneumoniae comprise repeating oligosaccharide units which may contain up to 8 sugar residues. For a review of the oligosaccharide units for the key Streptococcus pneumoniae serotypes see JONES, Christopher. Vaccines based on the cell surface carbohydrates of pathogenic bacteria. An. Acad. Bras. Ciênc., June 2005, vol. 77, no.2, p. 293-324. Table II ISSN 0001-3765. In one embodiment, a capsular saccharide antigen may be a full length polysaccharide, however in others it may be one oligosaccharide unit, or a shorter than native length saccharide chain of repeating oligosaccharide units. In one embodiment, all of the saccharides present in the vaccine are polysaccharides. Full length polysaccharides may be “sized” i.e. their size may be reduced by various methods such as acid hydrolysis treatment, hydrogen peroxide treatment, sizing by Emulsiflex® followed by a hydrogen peroxide treatment to generate oligosaccharide fragments or microfluidization.

The inventors have also noted that the focus of the art has been to use oligosaccharides for ease of conjugate production. The inventors have found that by using native or slightly sized polysaccharide conjugates, one or more of the following advantages may be realised: 1) a conjugate having high immunogenicity which is filterable, 2) the ratio of polysaccharide to protein in the conjugate can be altered such that the ratio of polysaccharide to protein (w/w) in the conjugate may be increased (which can have an effect on the carrier suppression effect), 3) immunogenic conjugates prone to hydrolysis may be stabilised by the use of larger saccharides for conjugation. The use of larger polysaccharides can result in more cross-linking with the conjugate carrier and may lessen the liberation of free saccharide from the conjugate. The conjugate vaccines described in the prior art tend to depolymerise the polysaccharides prior to conjugation in order to improve conjugation. The present inventors have found that saccharide conjugate vaccines retaining a larger size of saccharide can provide a good immune response against pneumococcal disease.

The immunogenic composition of the invention may thus comprise one or more saccharide conjugates wherein the average size (weight-average molecular weight; Mw) of each saccharide before conjugation is above 80 kDa, 100 kDa, 200 kDa, 300 kDa, 400 kDa, 500 kDa or 1000 kDa. In one embodiment the conjugate post conjugation should be readily filterable through a 0.2 micron filter such that a yield of more than 50, 60, 70, 80, 90 or 95% is obtained post filtration compared with the pre filtration sample.

For the purposes of the invention, “native polysaccharide” refers to a saccharide that has not been subjected to a process, the purpose of which is to reduce the size of the saccharide. A polysaccharide can become slightly reduced in size during normal purification procedures. Such a saccharide is still native. Only if the polysaccharide has been subjected to sizing techniques would the polysaccharide not be considered native.

The size of a native polysaccharide is for example between 250 kDa-2,000 kDa, 400-1,500 kDa 750 kDa-1,250 kDa, 300 kDa-600 kDa 500-1,000 kDa or 1,000-1,500 kDa with different serotypes having different sizes of native polysaccharide as will be appreciated by the skilled person.

For the purposes of the invention, “sized by a factor up to ×2” means that the saccharide is subject to a process intended to reduce the size of the saccharide but to retain a size more than half the size of the native polysaccharide. ×3, ×4 etc. are to be interpreted in the same way i.e. the saccharide is subject to a process intended to reduce the size of the polysaccharide but to retain a size more than a third, a quarter etc. the size of the native polysaccharide.

In an aspect of the invention, the immunogenic composition comprises Streptococcus pneumoniae saccharides from at least 10 serotypes conjugated to a carrier protein, wherein at least 1, 2, 3, 4, 5, 6, 7, 8, 9 or each S. pneumoniae saccharide is native polysaccharide.

In an aspect of the invention, the immunogenic composition comprises Streptococcus pneumoniae saccharides from at least 10 serotypes conjugated to a carrier protein, wherein at least 1, 2, 3, 4, 5, 6, 7, 8, 9 or each S. pneumoniae saccharide is sized by a factor up to ×2, ×3, ×4, ×5, ×6, ×7, ×8, ×9 or ×10. In one embodiment of this aspect, the majority of the saccharides, for example 6, 7, 8 or more of the saccharides are sized by a factor up to ×2, ×3, ×4, ×5, ×6, ×7, ×8, ×9 or x 10.

The molecular weight or average molecular weight of a saccharide herein refers to the weight-average molecular weight (Mw) of the saccharide measured prior to conjugation and is measured by MALLS.

The MALLS technique is well known in the art and is typically carried out as described in example 2. For MALLS analysis of pneumococcal saccharides, two columns (TSKG6000 and 5000PWxl) may be used in combination and the saccharides are eluted in water.

Saccharides are detected using a light scattering detector (for instance Wyatt Dawn DSP equipped with a 10 mW argon laser at 488 nm) and an inferometric refractometer (for instance Wyatt Otilab DSP equipped with a P100 cell and a red filter at 498 nm).

In an embodiment the S. pneumoniae saccharides are native polysaccharides or native polysaccharides which have been reduced in size during a normal extraction process.

In an embodiment, the S. pneumoniae saccharides are sized by mechanical cleavage, for instance by microfluidisation or sonication. Microfluidisation and sonication have the advantage of decreasing the size of the larger native polysaccharides sufficiently to provide a filterable conjugate. Sizing is by a factor of no more than ×20, ×10, ×8, ×6, ×5, ×4, ×3 or ×2.

In an embodiment, the immunogenic composition comprises S. pneumoniae conjugates that are made from a mixture of native polysaccharides and saccharides that are sized by a factor of no more than ×20. In one aspect of this embodiment, the majority of the saccharides, for example 6, 7, 8 or more of the saccharides are sized by a factor of up to ×2, ×3, ×4, ×5 or ×6.

In an embodiment, the immunogenic composition of the invention comprises the average size of the 19A saccharide is above 100 kDa, for example, between 110 and 700 kDa, 110-300, 120-200, 130-180, or 140-160 kDa. In an embodiment 19A is slightly sized by microfluidization, for example by a factor of up to ×2, ×3, ×4 or ×5. In an embodiment, the saccharide dose of the 19A conjugate is between 1 and 10 μg, 1 and 5 μg, or 1 and 3 μg of saccharide, optionally 3 μg of saccharide.

In an embodiment, the immunogenic composition of the invention comprises a 22F saccharide conjugate, wherein the average size of the 22F saccharide is above 100 kDa, optionally between 110 and 700 kDa, 110-300, 120-200, 130-180, or 150-170 kDa. In an embodiment, the 22F saccharide is sized by microfluidization, for example by a factor of up to ×2, ×3, ×4 or ×5. In an embodiment, the saccharide dose of the 19A conjugate is between 1 and 10 μg, 1 and 5 μg, or 1 and 3 μg of saccharide, optionally 3 μg of saccharide.

In an embodiment, the immunogenic composition of the invention comprises multiple saccharide conjugates wherein the average size of the saccharides is above 50 kDa. In an embodiment the average size of the serotype 1 saccharide is between 300 and 400 kDa. In an embodiment the average size of the serotype 4 saccharide is between 75 and 125 kDa. In an embodiment the average size of the serotype 5 saccharide is between 350 and 450 kDa. In an embodiment the average size of the serotype 6B saccharide is between 1000 and 1400 kDa. In an embodiment the average size of the serotype 7F saccharide is between 200 and 300 kDa. In an embodiment the average size of the serotype 9V saccharide is between 250 and 300 kDa. In an embodiment the average size of the serotype 14 saccharide is between 200 and 250 kDa. In an embodiment the average size of the serotype 23F saccharide is between 900 and 1000 kDa. In an embodiment the serotype(s) 5; 6A, 6B; 23F; 5 and 6A; 5 and 6B, 5 and 23F, 6A and 6B, 6A and 23F; 6B and 23F; 5, 6A and 6B; 5, 6A and 23F; 5, 6B and 23F or 5, 6A, 6B and 23F are conjugated as native sized saccharides, i.e with no dedicated sizing step included in the process.

In an embodiment, the immunogenic composition of the invention the saccharide dose of the capsular saccharide conjugates is between 1 and 10 μg, 1 and 5 μg, or 1 and 3 μg of saccharide per conjugate. For example, the composition comprises conjugates of serotypes 4, 18C, 19F and 22F (and optionally 19A) at dosages of 3 μg of saccharide per conjugate. For example, the immunogenic composition of the invention comprises conjugates of serotypes 1, 5, 6B, 7F, 9V, 14 and 23F (and optionally 6A and/or 3) at dosages of 1 μg of saccharide per conjugate.

In an embodiment, the Streptococcus pneumoniae saccharide is conjugated to the carrier protein via a linker, for instance a bifunctional linker. The linker is optionally heterobifunctional or homobifunctional, having for example a reactive amino group and a reactive carboxylic acid group, 2 reactive amino groups or two reactive carboxylic acid groups. The linker has for example between 4 and 20, 4 and 12, 5 and 10 carbon atoms. A possible linker is ADH. Other linkers include B-propionamido (WO 00/10599), nitrophenyl-ethylamine (Geyer et al (1979) Med. Microbiol. Immunol. 165; 171-288), haloalkyl halides (U.S. Pat. No. 4,057,685), glycosidic linkages (U.S. Pat. No. 4,673,574, U.S. Pat. No. 4,808,700), hexane diamine and 6-aminocaproic acid (U.S. Pat. No. 4,459,286). In an embodiment, ADH is used as a linker for conjugating saccharide from serotype 18C.

The saccharide conjugates present in the immunogenic compositions of the invention may be prepared by any known coupling technique. The conjugation method may rely on activation of the saccharide with 1-cyano-4-dimethylamino pyridinium tetrafluoroborate (CDAP) to form a cyanate ester. The activated saccharide may thus be coupled directly or via a spacer (linker) group to an amino group on the carrier protein. For example, the spacer could be cystamine or cysteamine to give a thiolated polysaccharide which could be coupled to the carrier via a thioether linkage obtained after reaction with a maleimide-activated carrier protein (for example using GMBS) or a haloacetylated carrier protein (for example using iodoacetimide [e.g. ethyl iodoacetimide HCl] or N-succinimidyl bromoacetate or SIAB, or SIA, or SBAP). Optionally, the cyanate ester (optionally made by CDAP chemistry) is coupled with hexane diamine or ADH and the amino-derivatised saccharide is conjugated to the carrier protein using carbodiimide (e.g. EDAC or EDC) chemistry via a carboxyl group on the protein carrier. Such conjugates are described in PCT published application WO 93/15760 Uniformed Services University and WO 95/08348 and WO 96/29094

Other suitable techniques use carbodiimides, carbiinides, hydrazides, active esters, norborane, p-nitrobenzoic acid, N-hydroxysuccinimide, S—NHS, EDC, TSTU. Many are described in WO 98/42721. Conjugation may involve a carbonyl linker which may be formed by reaction of a free hydroxyl group of the saccharide with CDI (Bethell et al J. Biol. Chem. 1979, 254; 2572-4, Hearn et al J. Chromatogr. 1981. 218; 509-18) followed by reaction of with a protein to form a carbamate linkage. This may involve reduction of the anomeric terminus to a primary hydroxyl group, optional protection/deprotection of the primary hydroxyl group' reaction of the primary hydroxyl group with CDI to form a CDI carbamate intermediate and coupling the CDI carbamate intermediate with an amino group on a protein.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508.

A further method involves the coupling of a cyanogen bromide (or CDAP) activated saccharide derivatised with adipic acid dihydrazide (ADH) to the protein carrier by Carbodiimide condensation (Chu C. et al Infect. Immunity, 1983 245 256), for example using EDAC.

In an embodiment, a hydroxyl group (optionally an activated hydroxyl group for example a hydroxyl group activated to make a cyanate ester [e.g. using CDAP]) on a saccharide is linked to an amino or carboxylic group on a protein either directly or indirectly (through a linker). Where a linker is present, a hydroxyl group on a saccharide is optionally linked to an amino group on a linker, for example by using CDAP conjugation. A further amino group in the linker for example ADH) may be conjugated to a carboxylic acid group on a protein, for example by using carbodiimide chemistry, for example by using EDAC. In an embodiment, the pneumococcal capsular saccharide(s) is conjugated to the linker first before the linker is conjugated to the carrier protein. Alternatively the linker may be conjugated to the carrier before conjugation to the saccharide.

A combination of techniques may also be used, with some saccharide-protein conjugates being prepared by CDAP, and some by reductive amination.

In general the following types of chemical groups on a protein carrier can be used for coupling/conjugation:

A) Carboxyl (for instance via aspartic acid or glutamic acid). In one embodiment this group is linked to amino groups on saccharides directly or to an amino group on a linker with carbodiimide chemistry e.g. with EDAC.
B) Amino group (for instance via lysine). In one embodiment this group is linked to carboxyl groups on saccharides directly or to a carboxyl group on a linker with carbodiimide chemistry e.g. with EDAC. In another embodiment this group is linked to hydroxyl groups activated with CDAP or CNBr on saccharides directly or to such groups on a linker; to saccharides or linkers having an aldehyde group; to saccharides or linkers having a succinimide ester group.
C) Sulphydryl (for instance via cysteine). In one embodiment this group is linked to a bromo or chloro acetylated saccharide or linker with maleimide chemistry. In one embodiment this group is activated/modified with bis diazobenzidine.
D) Hydroxyl group (for instance via tyrosine). In one embodiment this group is activated/modified with bis diazobenzidine.
E) Imidazolyl group (for instance via histidine). In one embodiment this group is activated/modified with bis diazobenzidine.
F) Guanidyl group (for instance via arginine).
G) Indolyl group (for instance via tryptophan).

On a saccharide, in general the following groups can be used for a coupling: OH, COOH or NH2. Aldehyde groups can be generated after different treatments known in the art such as: periodate, acid hydrolysis, hydrogen peroxide, etc.

### Direct Coupling Approaches:

Saccharide-OH+CNBr or CDAP->cyanate ester+NH2-Prot->conjugate
Saccharide-aldehyde+NH2-Prot->Schiff base+NaCNBH3->conjugate
Saccharide-COOH+NH2-Prot+EDAC->conjugate
Saccharide-NH2+COOH-Prot+EDAC->conjugate

### Indirect Coupling Via Spacer (Linker) Approaches:

Saccharide-OH+CNBr or CDAP->cyanate ester+NH2—NH2->saccharide—NH2+COOH-Prot+EDAC->conjugate
Saccharide-OH+CNBr or CDAP->cyanate ester+NH2 SH->saccharide—SH

+SH-Prot (native Protein with an exposed cysteine or obtained after modification of amino groups of the protein by SPDP for instance)->saccharide-S—S-Prot

Saccharide-OH+CNBr or CDAP->cyanate ester+NH2—SH->saccharide—SH

+maleimide-Prot (modification of amino groups)->conjugate

Saccharide-OH+CNBr or CDAP->cyanate ester+NH2 SH->Saccharide-SH+haloacetylated-Prot->Conjugate
Saccharide-COOH+EDAC+NH2—NH2->saccharide NH2+EDAC+COOH-Prot->conjugate
Saccharide-COOH+EDAC+NH2—SH->saccharide—SH+SH-Prot (native Protein with an exposed cysteine or obtained after modification of amino groups of the protein by SPDP for instance)->saccharide-S—S-Prot Saccharide-COOH+EDAC+NH2—SH->saccharide—SH+maleimide-Prot (modification of amino groups)->conjugate
Saccharide-COOH+EDAC+NH2—SH->Saccharide-SH+haloacetylated-Prot->Conjugate
Saccharide-Aldehyde+NH2—NH2->saccharide—NH2+EDAC+COOH-Prot->conjugate
Note: instead of EDAC above, any suitable carbodiimide may be used.

In summary, the types of protein carrier chemical group that may be generally used for coupling with a saccharide are amino groups (for instance on lysine residues), COOH groups (for instance on aspartic and glutamic acid residues) and SH groups (if accessible) (for instance on cysteine residues.

Optionally the ratio of carrier protein to S. pneumoniae saccharide is between 1:5 and 5:1; 1:2 and 2.5:1; 1:1 and 2:1 (w/w). In an embodiment, the majority of the conjugates, for example 6, 7, 8, 9 or more of the conjugates have a ratio of carrier protein to saccharide that is greater than 1:1, for example 1.1:1, 1.2:1, 1.3:1, 1.4:1, 1.5:1 or 1.6:1 (w/w).

In an embodiment, at least one S. pneumoniae saccharide is conjugated to a carrier protein via a linker using CDAP and EDAC. For example, 18C may be conjugated to a protein via a linker (for example those with two hydrazino groups at its ends such as ADH) using CDAP and EDAC as described above. When a linker is used, CDAP may be used to conjugate the saccharide to a linker and EDAC may then be used to conjugate the linker to a protein or, alternatively EDAC may be used first to conjugate the linker to the protein, after which CDAP may be used to conjugate the linker to the saccharide.

In general, the immunogenic composition of the invention may comprise a dose of each saccharide conjugate between 0.1 and 20 μg, 1 and 10 μg or 1 and 3 μg of saccharide.

In an embodiment, the immunogenic composition of the invention contains each S. pneumoniae capsular saccharide at a dose of between 0.1-20 μg; 0.5-10 μg; 0.5-5 μg or 1-3 μg of saccharide. In an embodiment, capsular saccharides may be present at different dosages, for example some capsular saccharides may be present at a dose of around or exactly 1 μg or some capsular saccharides may be present at a dose of around or exactly 3 μg. In an embodiment, saccharides from serotypes 3, 18C and 19F (or 4, 18C and 19F) are present at a higher dose than other saccharides. In one aspect of this embodiment, serotypes 3, 18C and 19F (or 4, 18C and 19F) are present at a dose of around or exactly 3 μg whilst other saccharides in the immunogenic composition are present at a dose of around or exactly 1 μg.

“Around” or “approximately” are defined as within 10% more or less of the given figure for the purposes of the invention.

In an embodiment, at least one of the S. pneumoniae capsular saccharides is directly conjugated to a carrier protein. Optionally the at least one of the S. pneumoniae capsular saccharides is directly conjugated by CDAP. In an embodiment, the majority of the capsular saccharides for example 5, 6, 7, 8, 9 or more are directly linked to the carrier protein by CDAP (see WO 95/08348 and WO 96/29094)

In an embodiment, the immunogenic composition of the invention comprises one or more unconjugated or conjugated S pneumoniae proteins. In an embodiment, the S. pneumoniae protein is added in unconjugated form, for example, it is present as a free protein in the composition.

In an embodiment, the immunogenic composition of the invention comprises at least or exactly 1, 2, 3 or 4 S. pneumoniae proteins are selected from Poly Histidine Triad family (PhtX), Choline Binding Protein family (CbpX), CbpX truncates, LytX family, LytX truncates, CbpX truncate-LytX truncate chimeric proteins, detoxified pneumolysin (Ply), PspA, PsaA, Sp128, Sp101, Sp130, Sp125 and Sp133. For example, the composition contains detoxified pneumolysin and/or PhtD. For example, the composition contains detoxified pneumolysin and PhtD and Sp128. For example, the composition contains detoxified pneumolysin and PhtD and Sp130.

The Pht (Poly Histidine Triad) family comprises proteins PhtA, PhtB, PhtD, and PhtE. The family is characterized by a lipidation sequence, two domains separated by a proline-rich region and several histidine triads, possibly involved in metal or nucleoside binding or enzymatic activity, (3-5) coiled-coil regions, a conserved N-terminus and a heterogeneous C terminus. It is present in all strains of pneumococci tested. Homologous proteins have also been found in other Streptococci and Neisseria. In one embodiment of the invention, the Pht protein of the invention is PhtD. It is understood, however, that the terms Pht A, B, D, and E refer to proteins having sequences disclosed in the citations below as well as naturally-occurring (and man-made) variants thereof that have a sequence homology that is at least 90% identical to the referenced proteins. Optionally it is at least 95% identical or at least 97% identical.

With regards to the PhtX proteins, PhtA is disclosed in WO 98/18930, and is also referred to Sp36. As noted above, it is a protein from the polyhistidine triad family and has the type II signal motif of LXXC. PhtD is disclosed in WO 00/37105, and is also referred to Sp036D. As noted above, it also is a protein from the polyhistidine triad family and has the type II LXXC signal motif. PhtB is disclosed in WO 00/37105, and is also referred to Sp036B. Another member of the PhtB family is the C3-Degrading Polypeptide, as disclosed in WO 00/17370. This protein also is from the polyhistidine triad family and has the type II LXXC signal motif. For example, an immunologically functional equivalent is the protein Sp42 disclosed in WO 98/18930. A PhtB truncate (approximately 79 kD) is disclosed in WO99/15675 which is also considered a member of the PhtX family. PhtE is disclosed in WO00/30299 and is referred to as BVH-3. Where any Pht protein is referred to herein, it is meant that immunogenic fragments or fusions thereof of the Pht protein can be used. For example, a reference to PhtX includes immunogenic fragments or fusions thereof from any Pht protein. A reference to PhtD or PhtB is also a reference to PhtDE or PhtBE fusions as found, for example, in WO0198334.

Pneumolysin is a multifunctional toxin with a distinct cytolytic (hemolytic) and complement activation activities (Rubins et al., Am. Respi. Cit Care Med, 153:1339-1346 (1996)). The toxin is not secreted by pneumococci, but it is released upon lysis of pneumococci under the influence of autolysin. Its effects include e.g., the stimulation of the production of inflammatory cytokines by human monocytes, the inhibition of the beating of cilia on human respiratory epithelial, and the decrease of bactericidal activity and migration of neutrophils. The most obvious effect of pneumolysin is in the lysis of red blood cells, which involves binding to cholesterol. Because it is a toxin, it needs to be detoxified (i.e., non-toxic to a human when provided at a dosage suitable for protection) before it can be administered in vivo. Expression and cloning of wild-type or native pneumolysin is known in the art. See, for example, Walker et al. (Infect Immun, 55:1184-1189 (1987)), Mitchell et al. (Biochim Biophys Acta, 1007:67-72 (1989) and Mitchell et al (NAR, 18:4010 (1990)). Detoxification of ply can be conducted by chemical means, e.g., subject to formalin or glutaraldehyde treatment or a combination of both (WO 04081515, PCT/EP2005/010258). Such methods are well known in the art for various toxins. Alternatively, ply can be genetically detoxified. Thus, the invention encompasses derivatives of pneumococcal proteins which may be, for example, mutated proteins. The term “mutated” is used herein to mean a molecule which has undergone deletion, addition or substitution of one or more amino acids using well known techniques for site directed mutagenesis or any other conventional method. For example, as described above, a mutant ply protein may be altered so that it is biologically inactive whilst still maintaining its immunogenic epitopes, see, for example, WO90/06951, Berry et al. (Infect Immun, 67:981-985 (1999)), WO99/03884 and WO 10/71986. The genetically detoxified pneumolysin may contains point mutations at amino acids 65 (threonine), 293 (glycine) and/or 428 (cysteine) as described in WO 10/71986.

As used herein, it is understood that the term “Ply” refers to mutated or detoxified pneumolysin suitable for medical use (i.e., non toxic).

Concerning the Choline Binding Protein family (CbpX), members of that family were originally identified as pneumococcal proteins that could be purified by choline-affinity chromatography. All of the choline-binding proteins are non-covalently bound to phosphorylcholine moieties of cell wall teichoic acid and membrane-associated lipoteichoic acid. Structurally, they have several regions in common over the entire family, although the exact nature of the proteins (amino acid sequence, length, etc.) can vary. In general, choline binding proteins comprise an N terminal region (N), conserved repeat regions (R1 and/or R2), a proline rich region (P) and a conserved choline binding region (C), made up of multiple repeats, that comprises approximately one half of the protein. As used in this application, the term “Choline Binding Protein family (CbpX)” is selected from the group consisting of Choline Binding Proteins as identified in WO97/41151, PbcA, SpsA, PspC, CbpA, CbpD, and CbpG. CbpA is disclosed in WO97/41151. CbpD and CbpG are disclosed in WO00/29434. PspC is disclosed in WO97/09994. PbcA is disclosed in WO98/21337. SpsA is a Choline binding protein disclosed in WO 98/39450. Optionally the Choline Binding Proteins are selected from the group consisting of CbpA, PbcA, SpsA and PspC.

An embodiment of the invention comprises CbpX truncates wherein “CbpX” is defined above and “truncates” refers to CbpX proteins lacking 50% or more of the Choline binding region (C). Optionally such proteins lack the entire choline binding region. Optionally, the such protein truncates lack (i) the choline binding region and (ii) a portion of the N-terminal half of the protein as well, yet retain at least one repeat region (R1 or R2). Optionally, the truncate has 2 repeat regions (R1 and R2). Examples of such embodiments are NR1×R2 and R1×R2 as illustrated in WO99/51266 or WO99/51188, however, other choline binding proteins lacking a similar choline binding region are also contemplated within the scope of this invention.

The LytX family is membrane associated proteins associated with cell lysis. The N-terminal domain comprises choline binding domain(s), however the LytX family does not have all the features found in the CbpA family noted above and thus for the present invention, the LytX family is considered distinct from the CbpX family. In contrast with the CbpX family, the C-terminal domain contains the catalytic domain of the LytX protein family. The family comprises LytA, B and C. With regards to the LytX family, LytA is disclosed in Ronda et al., Eur J Biochem, 164:621-624 (1987). LytB is disclosed in WO 98/18930, and is also referred to as Sp46. LytC is also disclosed in WO 98/18930, and is also referred to as Sp91. An embodiment of the invention comprises LytC.

Another embodiment comprises LytX truncates wherein “LytX” is defined above and “truncates” refers to LytX proteins lacking 50% or more of the Choline binding region. Optionally such proteins lack the entire choline binding region. Yet another embodiment of this invention comprises CbpX truncate-LytX truncate chimeric proteins (or fusions). Optionally this comprises NR1×R2 (or R1×R2) of CbpX and the C-terminal portion (Cterm, i.e., lacking the choline binding domains) of LytX (e.g., LytCCterm or Sp91Cterm). Optionally CbpX is selected from the group consisting of CbpA, PbcA, SpsA and PspC. Optionally, it is CbpA. Optionally, LytX is LytC (also referred to as Sp91). Another embodiment of the present invention is a PspA or PsaA truncate lacking the choline binding domain (C) and expressed as a fusion protein with LytX. Optionally, LytX is LytC.

With regards to PsaA and PspA, both are know in the art. For example, PsaA and transmembrane deletion variants thereof have been described by Berry & Paton, Infect Immun 1996 December; 64(12):5255-62. PspA and transmembrane deletion variants thereof have been disclosed in, for example, U.S. Pat. No. 5,804,193, WO 92/14488, and WO 99/53940.

Sp128 and Sp130 are disclosed in WO00/76540. Sp125 is an example of a pneumococcal surface protein with the Cell Wall Anchored motif of LPXTG (where X is any amino acid). Any protein within this class of pneumococcal surface protein with this motif has been found to be useful within the context of this invention, and is therefore considered a further protein of the invention. Sp125 itself is disclosed in WO 98/18930, and is also known as ZmpB—a zinc metalloproteinase. Sp101 is disclosed in WO 98/06734 (where it has the reference # y85993). It is characterized by a Type I signal sequence. Sp133 is disclosed in WO 98/06734 (where it has the reference # y85992). It is also characterized by a Type I signal sequence.

Examples of Moraxella catarrhalis protein antigens which can be included in a combination vaccine (especially for the prevention of otitis media) are: OMP106 [WO 97/41731 (Antex) & WO 96/34960 (PMC)]; OMP21 or fragments thereof (WO 0018910); LbpA &/or LbpB [WO 98/55606 (PMC)]; TbpA &/or TbpB [WO 97/13785 & WO 97/32980 (PMC)]; CopB [Helminen M E, et al. (1993) Infect. Immun. 61:2003-2010]; UspA1 and/or UspA2 [WO 93/03761 (University of Texas)]; OmpCD; HasR (PCT/EP99/03824); PiIQ (PCT/EP99/03823); OMP85 (PCT/EP00/01468); lipo06 (GB 9917977.2); lipo10 (GB 9918208.1); lipo11 (GB 9918302.2); lipo18 (GB 9918038.2); P6 (PCT/EP99/03038); D15 (PCT/EP99/03822); OmplA1 (PCT/EP99/06781); Hly3 (PCT/EP99/03257); and OmpE. Examples of non-typeable Haemophilus influenzae antigens or fragments thereof which can be included in a combination vaccine (especially for the prevention of otitis media) include: Fimbrin protein [(U.S. Pat. No. 5,766,608—Ohio State Research Foundation)] and fusions comprising peptides therefrom [eg LB1(f) peptide fusions; U.S. Pat. No. 5,843,464 (OSU) or WO 99/64067]; OMP26 [WO 97/01638 (Cortecs)]; P6 [EP 281673 (State University of New York)]; TbpA and/or TbpB; Hia; Hsf; Hin47; Hif; Hmw1; Hmw2; Hmw3; Hmw4; Hap; D15 (WO 94/12641); P2; and P5 (WO 94/26304).

The proteins of the invention may also be beneficially combined. By combined is meant that the immunogenic composition comprises all of the proteins from within the following combinations, either as carrier proteins or as free proteins or a mixture of the two. For example, in a combination of two proteins as set out hereinafter, both proteins may be used as carrier proteins, or both proteins may be present as free proteins, or both may be present as carrier and as free protein, or one may be present as a carrier protein and a free protein whilst the other is present only as a carrier protein or only as a free protein, or one may be present as a carrier protein and the other as a free protein. Where a combination of three proteins is given, similar possibilities exist. Combinations include, but are not limited to, PhtD+NR1×R2, PhtD+NR1×R2-Sp91Cterm chimeric or fusion proteins, PhtD+Ply, PhtD+Sp128, PhtD+PsaA, PhtD+PspA, PhtA+NR1×R2, PhtA+NR1×R2-Sp91Cterm chimeric or fusion proteins, PhtA+Ply, PhtA+Sp128, PhtA+PsaA, PhtA+PspA, NR1×R2+LytC, NR1×R2+PspA, NR1×R2+PsaA, NR1×R2+Sp128, R1×R2+LytC, R1×R2+PspA, R1×R2+PsaA, R1×R2+Sp128, R1×R2+PhtD, R1×R2+PhtA. Optionally, NR1×R2 (or R1×R2) is from CbpA or PspC. Optionally it is from CbpA. Other combinations include 3 protein combinations such as PhtD+NR1×R2+Ply, and PhtA+NR1×R2+PhtD. In one embodiment, the vaccine composition comprises detoxified pneumolysin and PhtD or PhtDE as carrier proteins. In a further embodiment, the vaccine composition comprises detoxified pneumolysin and PhtD or PhtDE as free proteins.

The present invention further provides a vaccine containing the immunogenic compositions of the invention and a pharmaceutically acceptable excipient.

The vaccines of the present invention may be adjuvanted, particularly when intended for use in an elderly population but also for use in infant populations. Suitable adjuvants include an aluminum salt such as aluminum hydroxide gel or aluminum phosphate or alum, but may also be other metal salts such as those of calcium, magnesium, iron or zinc, or may be an insoluble suspension of acylated tyrosine, or acylated sugars, cationically or anionically derivatized saccharides, or polyphosphazenes.

The adjuvant is optionally selected to be a preferential inducer of a TH1 type of response. Such high levels of Th1-type cytokines tend to favour the induction of cell mediated immune responses to a given antigen, whilst high levels of Th2-type cytokines tend to favour the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4 +ve T cell clones by Mosmann and Coffman (Mosmann, T. R. and Coffman, R. L. (1989) TH1 and TH2 cells: different patterns of lymphokine secretion lead to different functional properties. (Annual Review of Immunology, 7, p145-173). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of 11-4, IL-5, IL-6, IL-10. Suitable adjuvant systems which promote a predominantly Th1 response include: Monophosphoryl lipid A or a derivative thereof (or detoxified lipid A in general—see for instance WO2005107798), particularly 3-de-O-acylated monophosphoryl lipid A (3D-MPL) (for its preparation see GB 2220211 A); and a combination of monophosphoryl lipid A, optionally 3-de-O-acylated monophosphoryl lipid A, together with either an aluminum salt (for instance aluminum phosphate or aluminum hydroxide) or an oil-in-water emulsion. In such combinations, antigen and 3D-MPL are contained in the same particulate structures, allowing for more efficient delivery of antigenic and immunostimulatory signals. Studies have shown that 3D-MPL is able to further enhance the immunogenicity of an alum-adsorbed antigen [Thoelen et al. Vaccine (1998) 16:708-14; EP 689454-B1].

An enhanced system involves the combination of a monophosphoryl lipid A and a saponin derivative, particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210. In one embodiment the immunogenic composition additionally comprises a saponin, which may be QS21. The formulation may also comprise an oil in water emulsion and tocopherol (WO 95/17210). Unmethylated CpG containing oligonucleotides (WO 96/02555) and other immunomodulatory oligonucleotides (WO0226757 and WO03507822) are also preferential inducers of a TH1 response and are suitable for use in the present invention.

The vaccine preparations containing immunogenic compositions of the present invention may be used to protect or treat a mammal susceptible to infection, by means of administering said vaccine via systemic or mucosal route. These administrations may include injection via the intramuscular (IM), intraperitoneal (IP), intradermal (ID) or subcutaneous (SC) routes; or via mucosal administration to the oral/alimentary, respiratory, genitourinary tracts. Intranasal (IN) administration of vaccines for the treatment of pneumonia or otitis media is possible (as nasopharyngeal carriage of pneumococci can be more effectively prevented, thus attenuating infection at its earliest stage). Although the vaccine of the invention may be administered as a single dose, components thereof may also be co-administered together at the same time or at different times (for instance pneumococcal saccharide conjugates could be administered separately, at the same time or 1-2 weeks after the administration of the any bacterial protein component of the vaccine for optimal coordination of the immune responses with respect to each other). For co-administration, the optional Th1 adjuvant may be present in any or all of the different administrations. In addition to a single route of administration, 2 different routes of administration may be used. For example, saccharides or saccharide conjugates may be administered IM (or ID) and bacterial proteins may be administered IN (or ID). In addition, the vaccines of the invention may be administered IM for priming doses and IN for booster doses.

The content of protein antigens in the vaccine will typically be in the range 1-100 μg, optionally 5-50 μg, e.g. in the range 5-25 μg. Following an initial vaccination, subjects may receive one or several booster immunizations adequately spaced.

Vaccine preparation is generally described in Vaccine Design (“The subunit and adjuvant approach” (eds Powell M. F. & Newman M. J.) (1995) Plenum Press New York). Encapsulation within liposomes is described by Fullerton, U.S. Pat. No. 4,235,877.

The vaccines or immunogenic compositions of the present invention may be stored in solution or lyophilized. In an embodiment, the solution is lyophilized in the presence of a sugar acting as an amorphous lyoprotectant, such as sucrose, trehalose, glucose, mannose, maltose or lactose. In an embodiment, the solution is lyophilized in the presence of a sugar acting as an amorphous lyoprotectant, and a bulking agent providing improved cake structure such as glycine or mannitol. The presence of a crystalline bulking agent allows for shortening freeze-drying cycles, in the presence of high salt concentration. Examples of such mixtures for use in lyophilisation of the immunogenic compositions or vaccines of the invention include sucrose/glycine, trehalose/glycine, glucose/glycine, mannose/glycine, maltose/glycine, sucrose/mannitol/trehalose/mannitol, glucose/mannitol, mannose/mannitol and maltose/mannitol. Typically The molar ratio of the two constituents is optionally 1:1, 1:2, 1:3, 1:4, 1:5 or 1:6. Immunogenic compositions of the invention optionally comprise the lyophilisation reagents described above.

The above stabilising agents and mixtures of stabilising agents can further include a polymer capable of increasing the glass transition temperature (Tg′) of the formulation, such as poly(vinyl-pyrrolidone) (PVP), hydroxyethyl starch or dextran, or a polymer acting as a crystalline bulking agent such as polyethylene glycol (PEG) for example having a molecular weight between 1500 and 6000 and dextran.

The immunogenic compositions of the invention are optionally lyophilized and extemporaneously reconstituted prior to use. Lyophilizing may result in a more stable composition (vaccine) and may possibly lead to higher antibody titers in the presence of 3D-MPL and in the absence of an aluminum based adjuvant.

In one aspect of the invention is provided a vaccine kit, comprising a vial containing an immunogenic composition of the invention, optionally in lyophilised form, and further comprising a vial containing an adjuvant as described herein. It is envisioned that in this aspect of the invention, the adjuvant will be used to reconstitute the lyophilised immunogenic composition.

The present invention further provides an improved vaccine for the prevention or amelioration of Otitis media caused by Haemophilus influenzae by the addition of Haemophilus influenzae proteins, for example protein D in conjugated form. In addition, the present invention further provides an improved vaccine for the prevention or amelioration of pneumococcal infection in infants (e.g., Otitis media), by relying on the addition of one or two pneumococcal proteins as free or conjugated protein to the S. pneumoniae conjugate compositions of the invention. Said pneumococcal free proteins may be the same or different to any S. pneumoniae proteins used as carrier proteins. One or more Moraxella catarrhalis protein antigens can also be included in the combination vaccine in a free or conjugated form. Thus, the present invention is an improved method to elicit a (protective) immune response against Otitis media in infants.

In another embodiment, the present invention is an improved method to elicit a (protective) immune response in infants (defined as 0-2 years old in the context of the present invention) by administering a safe and effective amount of the vaccine of the invention [a paediatric vaccine]. Further embodiments of the present invention include the provision of the antigenic S. pneumoniae conjugate compositions of the invention for use in medicine and the use of the S. pneumoniae conjugates of the invention in the manufacture of a medicament for the prevention (or treatment) of pneumococcal disease.

In another embodiment, the present invention is an improved method to elicit a (protective) immune response in the elderly population (in the context of the present invention a patient is considered elderly if they are 50 years or over in age, typically over 55 years and more generally over 60 years) by administering a safe and effective amount of the vaccine of the invention, optionally in conjunction with one or two S. pneumoniae proteins present as free or conjugated protein, which free S. pneumoniae proteins may be the same or different as any S. pneumoniae proteins used as carrier proteins.

A further aspect of the invention is a method of immunising a human host against disease caused by S. pneumoniae and optionally Haemophilus influenzae infection comprising administering to the host an immunoprotective dose of the immunogenic composition or vaccine or kit of the invention.

A further aspect of the invention is an immunogenic composition of the invention for use in the treatment or prevention of disease caused by S. pneumoniae and optionally Haemophilus influenzae infection.

A further aspect of the invention is use of the immunogenic composition or vaccine or kit of the invention in the manufacture of a medicament for the treatment or prevention of diseases caused by S. pneumoniae and optionally Haemophilus influenzae infection.

The terms “comprising”, “comprise” and “comprises” herein are intended by the inventors to be optionally substitutable with the terms “consisting of”, “consist of” and “consists of”, respectively, in every instance.

Embodiments herein relating to “vaccine compositions” of the invention are also applicable to embodiments relating to “immunogenic compositions” of the invention, and vice versa.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1

### Conjugation Processes

The pneumococcal conjugates making up the seven valent Prevnar vaccine are made by conjugation of each serotype polysaccharide to a CRM197 carrier protein by a reductive amination process similar to that disclosed in WO 06/110381. Pneumococcal serotypes 4, 6B, 9V, 14, 18C, 19F and 23F all conjugated to CRM197 are present in the 7vCRM vaccine

Synflorix contains the same serotypes as 7vCRM, as well as additional serotypes 1, 5 and 7F. Serotypes 1, 4, 5, 6B, 7F, 9V, 14 and 23F polysaccharides are conjugated to protein D from non-typable Haemophilus influenzae, the 18C polysaccharide is conjugated to tetanus toxoid and the 19F polysaccharide is conjugated to diphtheria toxoid. The conjugation reactions use the cyanylation reagent CDAP and are essentially as described in WO 09/00824.

Serotype 18C was conjugated via an ADH linker using carbodiimide chamistry (EDAC) to activate tetanus toxoid with ADH and CDAP chemistry to couple polysaccharide 18C to the TT-ADH. The reaction was essentially as described in WO 09/00824.

Example 1a Conjugation of S. pneumoniae serotype 23 by CDAP

200 mg of microfluidized PS23F was dissolved in water until a concentration of 10 mg/ml was obtained. NaCl was added to this solution at a final concentration of 2M.

Sufficient CDAP solution (100 mg/ml freshly prepared in 5/50 v/v acetonitrile/WFI) was added to reach a CDAP:PS ratio of 0.75 mg/mg PS.

After 90 seconds, the pH was raised to pH 9.5 by addition of 0.1 M NaOH. 3 minutes later sufficient CRM197 (10 mg/ml in 0.15M NaCL) was added to reach a ratio of 1.5 (CRM197:PS (w/w)), the pH was maintained at pH 9.5. This solution was incubated for 1 hour at pH 9.5.

After this coupling step, 10 ml of 2M glycine solution was added to the mixture and the pH was adjusted to pH9.0 (the quenching pH). The solution was stirred for 30 minutes at room temperature. The conjugate was purified using a 5 μm filter followed by Sephacryl S400HR (XK50/100) which removes small molecules and unconjugated polysaccharides and protein. The flow rate was fixed at 150 ml/hour. Elution was achieved using 150 mM NaCl. The fractions of interest were pooled and filtered using Milipack 20. The resulting conjugate had a final CRM197/PS ratio (w/w) of 1.35/1 (w/w).

### Example 2

### Clinical Trial Data Comparing 7vCRM197 (Prevnar) and PHiD-CV (Synflorix) Vaccines

A comparision was made of the immune responses elicited against S. pneumoniae 19F and 19A by 7vCRM197 and PHiD-CV. Both vaccine contain a 19F conjugate, which is conjugated to the non-toxic diphtheria toxin CRM197 by reductive amination in 7vCRM197 and is conjugated to diphtheria toxoid using the cyanylation reagent CDAP in PHiD-CV. Neither vaccine contains a 19A conjugate, however, the similarity of structure between 19A and 19F allows some generation of cross-reactive antibodies to 19A following immunisation with 19F.

### Serum Samples

Data from three primary vaccination studies (001, 011 and 012)⁸⁻¹⁰ comparing 7vCRM and PHiD-CV administered to infants in a three-dose primary series were reviewed (see Table 1 for primary study details). Data from booster studies associated with each primary study were also analysed (007⁸, 017⁹ and 018) (see Table 2 for booster study details). In all studies, blood samples were collected 1 month post-dose 3 (primary studies) and 1 month post-booster dose (booster studies).

### Immunological Assays

Antibody responses against serotype 19F and the related serotype 19A were evaluated using an ELISA with a 22F-preincubation step developed by GSK Biologicals (GSK-22F-ELISA, in which the heterologous serotype 22F polysaccharide is added to remove non-serotype-specific and non-opsonic antibodies.^{6,7}

The assay sensitivity for the 22F-inhibition ELISA was 0.05 μg/mL IgG.

Functional antibody responses were evaluated using GSK and THL OPA assays, which uses a modification of the HL-60 cell WHO reference method.^{2,4}

The OPA titre was defined as the reciprocal of the lowest serum dilution that induced

≧50% bacterial cell death compared to the control wells, and a titre of (a serum dilution of 1:8) was used as the threshold for this assay.^{2,4}

In addition, sera with an antibody concentration of pg/mL against serotype 19F (obtained from Dr. David Goldblatt, Institute of Child Health, UK) and from unimmunised healthy adults (obtained from the National Institutes of Health blood bank, Bethesda, Md.) were used for binding and inhibition by different forms of serotype 19F antigens (the unconjugated native polysaccharide and the conjugated 19F using reductive amination and cyanylation).

### Statistical Analysis

The percentage of serum samples with an ELISA IgG antibody concentration≧0.2 μg/mL, and the percentage of serum samples with an OPA titre≧8 were calculated with 95% confidence intervals.

The geometric mean OPA titres (GMTs) and geometric mean OPA/ELISA ratios (GMRs) were calculated in order to evaluate functional activity compared with antibody titre alone. Bridging GSK and THL OPA assays was conducted to assess the level of variability in OPA responses in different laboratories.

### Results

Data for at least one serotype (either 19A or 19F) were available for a total 709 infants primed with PHiD-CV and 331 infants primed with 7vCRM (Table 1) and for a total of 690 infants boosted with PHiD-CV and 292 infants boosted with 7vCRM (Table 2).

### Immunogenicity

### Serotype 19F—Primary Vaccination

Across the three primary studies, 87.7-99.3% of infants receiving PHiD-CV achieved OPA titres≧8 against serotype 19F, compared with 91.3-92.1% of infants receiving 7vCRM (FIG. 2).

OPA GMTs and OPA/ELISA GMRs for serotype 19F were higher in infants receiving PHiD-CV (Table 1).

### Serotype 19F—Booster Vaccination

In the booster studies, OPA titres≧8 against serotype 19F were achieved in 94.9-100.0% of infants receiving PHiD-CV compared with 92.5-98.5% of infants receiving 7vCRM (FIG. 2).

OPA GMTs for serotype 19F were higher in infants receiving PHiD-CV and OPA/ELISA GMRs were within the same range for both vaccines (Table 2).

### Serotype 19A—Primary Vaccination

An OPA titre against the cross-reactive serotype 19A was achieved in 19.6-28.7% of infants receiving PHiD-CV compared with 0.0-3.4% of infants receiving 7vCRM (FIG. 3).

OPA GMTs for serotype 19A were also higher in infants receiving PHiD-CV (Table 1).

Serotype 19A—booster vaccination

OPA titres≧8 against the cross-reactive serotype 19A were achieved in 37.7-69.2% of infants receiving PHiD-CV compared with 24.0-37.5% of infants receiving 7vCRM (FIG. 3).

OPA GMTs for serotype 19A were generally higher in infants receiving PHiD-CV (Table 2).

### Bridging OPA Assays

19F OPA results were comparable between GSK and THL when assessed in bridging studies whilst the 19A OPA assay at GSK appears to underestimate the responses.

A significant proportion of 19F-conjugate immunised children turned seropositive for 19A OPA at THL whilst being seronegative at GSK.

### Conclusions

PHiD-CV, containing 19F-DT prepared via cyanylation conjugation chemistry, induced higher levels of functional antibodies against serotype 19F, as measured by OPA assay, compared with 7vCRM vaccination containing 19F—CRM₁₉₇ prepared by reductive amination.

The higher OPA responses against serotype 19F achieved using cyanylation-conjugation also resulted in improved OPA responses against the cross-reactive serotype 19A for PHiD-CV compared with 7vCRM.

Bridging data suggest that the GSK 19A OPA assay underestimates serotype 19A OPA responses.

TABLE 1
Geometric mean opsonophagocytic activity (OPA) assay titres (GMTs) and geometric
mean OPA/ELISA ratios (GMRs) against pneumococcal serotype 19F and cross-reactive
serotype 19A following PHiD-CV or 7vCRM primary immunisation.

| | | | Serotype 19F | | | | Serotype 19A | |
|---|---|---|---|---|---|---|---|---|
| Study | | | OPA GMT | | OPA/ELISA GMR | | OPA GMT | |
| (vaccination | Study | PCV | | GMT | | GMR | | GMT |
| schedule) | number | vaccine | N | (95% CI) | N′ | (95% CI) | N | (95% CI) |
| Study 001⁸ | 105553/ | PHiD-CV^{†} | 268 | 148.6 | 235 | 108.1 | 260 | 8.6 |
| (2, 3, 4 mo) | NCT00307554 | | | (117.8-187.5) | | (93.31-125.25) | | (7.1-10.5) |
| | | 7vCRM^{†} | 89 | 52.0 | 82 | 16.2 | 89 | 4.5 |
| | | | | (38.9-69.4) | | (12.71-20.63) | | (3.9-5.3) |
| Study 011⁹ | 107005/ | PHiD-CV^{‡} | 159 | 261.0 | 150 | 78.3 | 101 | 7.1 |
| (2, 4, 6 mo) | NCT00334334 | | | (200.9-339.0) | | (64.3-95.4) | | (5.6-9.0) |
| | | 7vCRM^{‡} | 147 | 52.0 | 133 | 23.6 | 97 | 4.1 |
| | | | | (40.8-66.4) | | (19.7-28.3) | | (3.9-4.2) |
| Study 012 | 107007/ | PHiD-CV^{#} | 143 | 337.8 | 142 | 66.6 | 143 | 10.1 |
| Poland¹⁰ | NCT00344318 | | | (262.9-434.1) | | (55.4-80.0) | | (7.8-13.1) |
| (2, 4, 6 mo) | | 7vCRM^{#} | 49 | 35.9 | 45 | 16.2 | 49 | 4.0 |
| | | | | (25.7-50.1) | | (12.0-21.9) | | (4.0-4.0) |
| Study 012 | 107007/ | PHiD-CV* | 139 | 1121.7 | 137 | 109.3 | 137 | 10.6 |
| Philippines¹⁰ | NCT00344318 | | | (931.5-1350.6) | | (93.3-128.0) | | (7.9-14.2) |
| (6, 10, 14 wks) | | 7vCRM* | 46 | 81.6 | 42 | 22.3 | 44 | 4.2 |
| | | | | (53.0-125.5) | | (16.6-29.9) | | (3.8-4.7) |

GMT, geometric mean OPA titres;
GMR, geometric mean of ratios opsonophagocytic titres/ELISA antibody concentrations;
OPA, opsonophagocytic activity assay;
N = number of infants with available OPA results for serotype 19F or for the cross-reactive serotype 19A
N′ = number of infants with ELISA concentrations ≧0.05 μg/mL and OPA titres ≧8
Co-administered vaccines:
^{†}DTPa-HBV-IPV/Hib,
^{‡}DTPa-HBV-IPV + Hib-MenC,
^{#}DTPw-HBV/Hib + IPV,
*DTPw-HBV/Hib + OPV

TABLE 2
Geometric mean opsonophagocytic activity (OPA) assay titres (GMTs) and geometric
mean OPA/ELISA ratios (GMRs) against pneumococcal serotype 19F and cross-reactive
serotype 19A following PHiD-CV or 7vCRM booster immunisation.

| | | | Serotype 19F | | | | Serotype 19A | |
|---|---|---|---|---|---|---|---|---|
| Study | | | OPA GMT | | OPA/ELISA GMR | | OPA GMT | |
| (vaccination | Study | PCV | | GMT | | GMR | | GMT |
| schedule) | number | vaccine | N | (95% CI) | N′ | (95% CI) | N | (95% CI) |
| Study 007⁸ | 107046/ | PHiD-CV^{†} | 293 | 624.3 | 278 | 123.0 | 287 | 29.2 |
| (12-18 mo) | NCT00370396 | | | (509.7-764.7) | | (107.9-140.3) | | (22.3-38.3) |
| | | 7vCRM^{†} | 80 | 287.8 | 72 | 112.4 | 76 | 11.1 |
| | | | | (190.8-434.3) | | (83.2-151.9) | | (7.3-17.0) |
| Study 017⁹ | 109507/ | PHiD-CV^{‡} | 139 | 551.3 | 139 | 84.2 | 138 | 18.2 |
| (11-18 mo) | NCT00463467 | | | (443.2-685.9) | | (69.8-101.7) | | (12.8-26.0) |
| | | 7vCRM^{‡} | 133 | 321.3 | 131 | 87.6 | 125 | 9.1 |
| | | | | (251.2-411.0) | | (71.8-107.0) | | (6.8-12.2) |
| Study 018 | 109509/ | PHiD-CV^{#} | 122 | 1059.8 | 118 | 91.1 | 115 | 70.7 |
| Poland | NCT00547248 | | | (808.2-1389.7) | | (82.1-101.1) | | (45.3-110.4) |
| (12-18 mo) | | 7vCRM^{#} | 37 | 471.0 | 36 | 83.8 | 32 | 20.3 |
| | | | | (270.0-821.8) | | (58.9-119.2) | | (8.8-46.8) |
| Study 018 | 109509/ | PHiD-CV* | 136 | 2016.0 | 135 | 97.8 | 133 | 89.3 |
| Philippines | NCT00547248 | | | (1609.0-2526.0) | | (86.5-110.5) | | (58.9-135.4) |
| (12-18 mo) | | 7vCRM* | 42 | 473.2 | 39 | 91.4 | 40 | 8.7 |
| | | | | (263.5-849.9) | | (69.7-119.8) | | (5.4-14.2) |

GMT, geometric mean OPA titres;
GMR, geometric mean of ratios opsonophagocytic titres/ELISA antibody concentrations;
OPA, opsonophagocytic activity assay;
N, number of infants with available OPA results for serotype 19F or for the cross-reactive serotype 19A
N′, number of infants with ELISA concentrations ≧0.05 μg/mL and OPA titres ≧8
Co-administered vaccines:
^{†}DTPa-HBV-IPV/Hib,
^{‡}DTPa-HBV-IPV + Hib-MenC,
^{#}DTPw-HBV/Hib + IPV,
*DTPw-HBV/Hib + OPV

### Example 3

### Oxidation of 23F and 6B Using Periodate

Polysaccharides (PS) 23F or 6B were dissolved in 100 mM KH₂PO₄ (pH 7.4), 10 mM KH₂PO₄ or WFI, to form solutions of 2 mgPS/ml. The solution was incubated for 2 hours under agitation at room temperature. After this time the pH was adjusted to pH 6.0 with 1 MHCl. Periodate was added as a powder or in liquid form (10 mg/ml in WFI) in various amounts to achieve a range of molar ratios (table 3). The solutions were incubated for 17 hours at room temperature (20-25° C.), after which time the samples were dialyzed or diafiltered against WFI.

High performance gel filtration chromatography coupled with refractive index and multiangle laser lights scattering (MALLS-Dawn EOS) detectors was used to measure the molecular weight and the sample concentration applying Zimm model. Size exclusion media (TSK5000PWXL-Tosoh) was used to profile the molecular size distribution of the polysaccharide (elution 0.5 ml/min in NaCl 0.2M-NaN3 0.02%).

Table 3 and FIG. 4 describe the results of these experiments. These demonstrate that for the 23F saccharide substantial sizing occurs on oxidation using high molar equivalents of periodate in 100 mM phosphate buffer. This sizing effect can be reduced by reducing the concentration of phosphate buffer or the molar equivalents of periodate used.

TABLE 3

| 23F | | | | 6B | | | |
|---|---|---|---|---|---|---|---|
| | molar | | | | molar | | |
| | equivalent of | | Size | | equivalent | | Size |
| Sample | periodate | Buffer | (KDa) | Sample | of periodate | buffer | (KDa) |
| 23F native | 0 | Water | 861 | 6B | 0 | 10 mM | 1022 |
| | | | | | | phosphate | |
| 23F native | 0 | 10 mM phosphate | 847 | 6B | 0.1 | 10 mM | 975 |
| | | | | | | phosphate | |
| 23F native | 0 | 100 mM | 860 | 6B | 0.2 | 10 mM | 990 |
| | | phosphate | | | | phosphate | |
| 23F ATCC | 0 | 100 mM | 1655 | 6B | 0.3 | 10 mM | 961 |
| native | | phosphate | | | | phosphate | |
| 23F | 1 | 100 mM | <1 | 6B | 0.75 | 10 mM | 868 |
| | | phosphate | | | | phosphate | |
| 23F | 1 | Water | 36 | | | | |
| 23F | 1.2 | 100 mM | <1 | | | | |
| | | phosphate | | | | | |
| 23FATCC | 1 | 100 mM | 2 | | | | |
| | | phosphate | | | | | |
| 23FATCC | 0.125 | 100 mM | 39 | | | | |
| | | phosphate | | | | | |
| 23F | 0.1 | 10 mM phosphate | 466.9 | | | | |
| 23F | 0.15 | 10 mM phosphate | 398.5 | | | | |
| 23F | 0.2 | 10 mM phosphate | 336 | | | | |
| 23F | 0.5 | 10 mM phosphate | 179.1 | | | | |

### Reductive Amination

1 g of PS23F was dissolved in 500 ml of 10 mM KH₂PO₄, pH 7.15. This solution was incubated at room temperature for two hours. The pH was adjusted to 6.0M with 1M HCl. 111 mg of periodate (NalO₄, 0.4 molar equivalents of periodate) was added to the PS23F solution, and the solution was incubated for 17 hours in the dark at room temperature to oxidise PS23F. The solution was then diafiltered against WFI (Pellicon 2, 1000 cm²).

The oxidised PS23F was lyophilised with the CRM197 protein (at a CRM/PS ratio (w/w): 0.625) in the presence of 3% sucrose (w/v).

900 mg of the lyophilised PS23F/CRM197 mixture was solubilised by addition of 350 ml of DMSO solvent and incubating for 2 hours at 20° C. To reduce the PS23F/CRM197 mixture 1 molar equivalent of NaBH₃CN was added (735 μl of a solution of 100 mg/ml in WFI). The solution was incubated for a further 40 hours at room temperature under agitation. After this time 2 molar equivalent of NaBH₄ (100 mg/ml in WFI) was added and the solution incubated for 4 hours at room temperature. 2200 ml of 150 mM NaCl was added before diafiltration (cut-off 100 kDa) and purification by DEAE (XK50). The fractions of interest were pooled and filtered through a 0.22 μm filter.

### Example 4

### Comparison of the Immunogenicity of PS23F—CRM Conjugates, Conjugated Using Reductive Amination with PS23F—CRM Conjugates Conjugated Using CDAP Chemistry

### Immunogenicity Measured in a Guinea Pig Model

Female guinea pigs were immunized intramuscularly three times (at days 0, 14 and 28) with 0.25 μg of the PS23F—CRM197 conjugates. Animals were bled on day 42 and the antibody response directed against PS23F was measured by ELISA and OPA. Results are shown in FIG. 5.

A significantly higher antibody response was induced in the guinea pigs after immunisation with PS23F—CRM197 conjugated by reductive amination than PS23F—CRM197 conjugated by CDAP chemistry as seen in FIG. 5.

### Example 5

### Comparison of the Immunogenicity of PS6B-CRM Conjugates, Conjugated Using Reductive Amination with PS6B-CRM or PS6B-PD Conjugates Conjugated Using CDAP Chemistry

### Preclinical Studies:

Groups of 40 female Balb/c mice (4 weeks-old) were immunized intramuscularly three times at days 0, 14 and 28 with 0.1 μg of PS6B conjugates produced by reductive aminiation or CDAP chemistry formulated on AlPO4. PS6B-PD was used as benchmark. Mice were bled on day 42 and the antibody response directed against each antigen was measured by ELISA and OPA.

Groups of 20 female guinea pig (150 gr from Hartley) were immunized intramuscularly three times at days 0, 14 and 28 with 0.25 μg of PS6B conjugates produced by amino reductive or CDAP chemistry formulated on AlPO4. PS6B-PD was used as benchmark. Guinea pigs were bled on day 42 and the antibody response directed against each antigen was measured by ELISA and OPA.

Four different conjugates of PS6B-CRM made by reductive amination and one made using CDAP were used. The polysaccharides were microfluidized to two different molecular weights. The properties of the conjugates were:

| Conjugate | PS size | CRM/PS ratio (w/w) |
|---|---|---|
| PS06B-CRM 122 | 84 kDa | 1.09/1 |
| PS06B-CRM 123 | 84 kDa | 3/1 |
| PS06B-CRM 124 | 350 kDa | 1.6/1 |
| PS06B-CRM 125 | 350 kDa | 2.9/1 |

Mouse and Guinea Pig OPA

Serum samples were heated for 45 min at 56° C. to inactivate any remaining endogenous complement. Twenty-five microlitres aliquots of each 1:2 diluted serum sample was two-fold serially diluted in 25 μl OPA buffer (HBSS-14.4% inactivated FBS) per well of a 96-well round bottom microtitre plate. Subsequently, 25 μl of a mixture of activated HL-60 cells (1×10⁷ cells/ml), freshly thawed pneumococcal working seed and freshly thawed baby rabbit complement in an e.g. 4/2/1 ratio (v/v/v) were added to the diluted sera to yield a final volume of 50 μl. The assay plate was incubated for 2 h at 37° C. with orbital shaking (210 rpm) to promote the phagocytic process. The reaction was stopped by laying the microplate on ice for at least 1 min. A 20 μl aliquot of each well of the plate was then transferred into the corresponding well of a 96-well flat bottom microplate and 50 μl of Todd-Hewitt Broth-0.9% agar was added to each well. After overnight incubation at 37° C. and 5% CO2, pneumococcal colonies appearing in the agar were counted using an automated image analysis system (KS 400, Zeiss, Oberkochen, Germany). Eight wells without serum sample were used as bacterial controls to determine the number of pneumococci per well. The mean number of CFU of the control wells was determined and used for the calculation of the killing activity for each serum sample. The OPA titre for the serum samples was determined by the reciprocal dilution of serum able to facilitate 50% killing of the pneumococci. The opsonophagocytic titre was calculated by using a 4-parameter curve fit analysis.

TABLE 4
Preclinical mouse model - ELISA GMC in μg/ml and % responders

| | G1 | G2 | G3 | G4 | G5 | G6 |
|---|---|---|---|---|---|---|
| Subject/Result | PS06B-CRM122 | PS06B-CRM123 | PS06B-CRM124 | PS06B-CRM125 | PS06B-CRM003 | PS06B-PD |
| | (R: 1/1, PS 84 | (R: 3/1, PS 84 kDa) | (R: 1.5/1, PS 350 kDa) | (R: 2.9/1, PS 350 kDa) | (CDAP) | |
| GMC (UG-ML) | 0.83 | 0.37 | 1.18 | 0.64 | 0.31 | 0.10 |
| Responders (%) | 31/40 | 26/40 | 33/40 | 29/40 | 29/40 | 15/40 |

TABLE 5
Preclinical guinea pig model - ELISA GMC in μg/ml and % responders

| | G1 | G2 | G3 | G4 | G5 | G6 |
|---|---|---|---|---|---|---|
| Subject/Result | PS06B-CRM122 | PS06B-CRM123 | PS06B-CRM124 | PS06B-CRM125 | PS06B-CRM003 | PS06B-PD |
| | (R: 1/1, PS 84 kDa) | (R: 3/1, PS84 kDa) | (R: 1.5/1, | (R: 2.9/1, PS 350 kDa) | (CDAP) | |
| | | | PS0350 kDa) | | | |
| GMC (UG-ML) | 3.51 | 7.70 | 2.84 | 19.93 | 3.70 | 1.55 |
| Responders (%) | 20/20 | 20/20 | 20/20 | 20/20 | 20/20 | 20/20 |

1. An immunogenic composition comprising at least 2 different S. pneumoniae capsular saccharides, wherein one or more is/are selected from a first group consisting of serotypes 1, 3, 19A and 19F which is/are linked to a protein carrier(s) either directly or indirectly through a chemistry other than reductive amination, and one or more different saccharides is/are selected from a second group consisting of serotypes 4, 5, 6A, 6B, 7F, 9V, 14, 18C and 23F which is/are linked to a protein carrier(s) by reductive amination.

2. The immunogenic composition of claim 1 comprising a S. pneumoniae capsular saccharide from serotype 1 linked to a protein carrier through a chemistry other than reductive amination wherein the saccharide from serotype 1 is conjugated to the protein carrier through a cyanylation chemistry.

3.-7. (canceled)

8. The immunogenic composition of claim 1 comprising a S. pneumoniae capsular saccharide from serotype 19A linked to a protein carrier through a chemistry other than reductive amination.

9.-10. (canceled)

11. The immunogenic composition of claim 1 comprising a S. pneumoniae capsular saccharide from serotype 19F conjugated linked to a protein carrier through a chemistry other than reductive amination.

12.-13. (canceled)

14. The immunogenic composition of claim 1 comprising a S. pneumoniae capsular saccharide from serotype 4 linked to a protein carrier by reductive amination.

15. The immunogenic composition of claim 1 comprising a S. pneumoniae capsular saccharide from serotype 5 linked to a protein carrier by reductive amination.

16. The immunogenic composition of claim 1 comprising a S. pneumoniae capsular saccharide from serotype 6A linked to a protein carrier by reductive amination.

17. The immunogenic composition of claim 1 comprising a S. pneumoniae capsular saccharide from serotype 6B linked to a protein carrier by reductive amination.

18. The immunogenic composition of claim 1 comprising a S. pneumoniae capsular saccharide from serotype 7F linked to a protein carrier by reductive amination.

19. The immunogenic composition of claim 1 comprising a S. pneumoniae capsular saccharide from serotype 9V linked to a protein carrier by reductive amination.

20. The immunogenic composition of claim 1 comprising a S. pneumoniae capsular saccharide from serotype 14 linked to a protein carrier by reductive amination.

21. The immunogenic composition of claim 1 comprising a S. pneumoniae capsular saccharide from serotype 18C linked to a protein carrier by reductive amination.

22. The immunogenic composition of claim 1 comprising a S. pneumoniae capsular saccharide from serotype 23F linked to a protein carrier by reductive amination.

23.-27. (canceled)

28. The immunogenic composition of claim 1 wherein the carrier protein is selected from the group consisting of tetanus toxoid, diphtheria toxoid, CRM197 and Protein D.

29.-77. (canceled)

78. The immunogenic composition of claim 1 which further comprises one or more unconjugated or conjugated S. pneumoniae proteins.

79.-85. (canceled)

86. The immunogenic composition according to claim 1 which further comprises an adjuvant.

87.-119. (canceled)

120. A method of eliciting an immune response in a mammal comprising administering to the mammal the immunogenic composition of claim 1.
